(19) United States
(12) Patent Application Publication
(10) Pub. No.: US 2017/0368474 A1
(43) Pub. Date: Dec. 28, 2017

(54) METHOD AND APPARATUS FOR EXTRACTING OIL FROM FOOD WASTE

(71) Applicant: Research Foundation of the City University of New York, New York, NY (US)

(72) Inventor: Lawrence M. Pratt, Stratford, CT (US)

(73) Assignee: Research Foundation of the City University of New York, New York, NY (US)

(21) Appl. No.: 15/533,503

(22) PCT Filed: Dec. 14, 2015
(86) PCT No.: PCT/US15/65542

§ 371 Date: Jun. 6, 2017

Related U.S. Application Data
(60) Provisional application No. 62/090,995, filed on Dec. 12, 2014.

(51) Int. Cl. B01D 3/38; C12M 1/00; B01D 17/02; C11B 13/00; B09B 3/00

(52) U.S. Cl. CPC B01D 3/38; C12M 43/02; B01D 17/0217; B09B 3/0016; C12M 21/12; C11B 13/00

(57) ABSTRACT

A method and device for extracting oil from food waste is disclosed. Food waste is introduced into an elongated cylinder that is immersed under hot water. A drive shaft is actuated that simultaneously presses the food waste against an interior wall and moves the food waste through the cylinder. The content of the cylinder is subjected to steam treatment using a steam inlet in the cylinder. The combination of hot water, steam treatment and pressing recovers a high content of oil from the food waste.

### CROSS-REFERENCE TO RELATED APPLICATIONS

This application is a non-provisional of U.S. Patent Application Ser. No. 62/090,995 (filed Dec. 12, 2014), the entirety of which is incorporated herein by reference.

### STATEMENT OF FEDERALLY SPONSORED RESEARCH OR DEVELOPMENT

This invention was made in part with Government support under grant number INT 1129273 awarded by the National Science Foundation. The government has certain rights in the invention.

### BACKGROUND OF THE INVENTION

The subject matter disclosed herein relates to the extraction of oils from food waste for subsequent use as biofuel. High petroleum prices and concerns over global warming have increased the demand for biofuel raw materials. Among the most popular biofuels is biodiesel, consisting of the methyl or ethyl esters of fatty acids. These esters have been made from waste vegetable oil and used cooking oil, as well as virgin vegetable oils and animal fats. The major limitation of waste cooking oils and animal fats is that the demand greatly exceeds the supply. Virgin vegetable oil is more expensive than petroleum diesel, and growing sufficient quantities of oil crops would require so much arable land as to interfere with food production. Algae is another potential source of biodiesel and hydrocarbon diesel, but thus far, production costs make it more expensive than petroleum diesel. Alternative sources are therefore desired.

The discussion above is merely provided for general background information and is not intended to be used as an aid in determining the scope of the claimed subject matter.

### BRIEF DESCRIPTION OF THE INVENTION

A method and device for extracting oil from food waste is disclosed. Food waste is introduced into an elongated cylinder that is immersed under hot water. A drive shaft is actuated that simultaneously presses the food waste against an interior wall and moves the food waste through the cylinder. The content of the cylinder is subjected to steam treatment using a steam inlet in the cylinder. The combination of hot water, steam treatment and pressing recovers a high content of oil from the food waste. An advantage that may be realized in the practice of some disclosed embodiments of the method is that a high content of oil that be consistently recovered in relatively small processing times.

In a first embodiment, a method for extracting oil from food waste is provided. The method comprises steps of introducing food waste into an elongated cylinder that has a length, the elongated cylinder disposed in a reservoir and being at least partially immersed under water, the cylinder comprising a proximate end and a distal end, the food waste being introduced to the proximate end; at least one pore at the distal end, the at least one pore being on a sidewall; a steam inlet at the distal end that introduces steam into the cylinder; a drive shaft that extends the length of the cylinder. The method further comprises actuating the drive shaft with a motor while the water is at a temperature of at least 80° C. and steam is being supplied to the cylinder through the steam inlet, the drive shaft simultaneously pressing the food waste against an interior wall of the cylinder and moving the food waste from the proximate end to the distal end, thereby extracting an oil from the food waste; extruding the oil through the at least one pore, the oil thereafter floating to a surface of the water; and skimming the oil off the surface of the water.

In a second embodiment, a method for extracting oil from food waste is provided. The method comprising steps of introducing food waste into an elongated cylinder that has a length, the elongated cylinder disposed in a reservoir and being at least partially immersed under water, the cylinder comprising a proximate end and a distal end, the food waste being introduced to the proximate end; at least one pore at the distal end, the at least one pore being on a sidewall; a steam inlet at the distal end that introduces steam into the cylinder; a drive shaft that extends the length of the cylinder. The method further comprises actuating the drive shaft with a motor while the water is at a temperature of at least 80° C. and steam is being supplied to the cylinder through the steam inlet, the drive shaft simultaneously pressing the food waste against an interior wall of the cylinder and moving the food waste from the proximate end to the distal end, thereby extracting an oil from the food waste, wherein the drive shaft has an auger, the drive shaft being configured to rotate the auger and move the food waste from the proximate end to the distal end; extruding the oil through the at least one pore, the oil thereafter floating to a surface of the water; and skimming the oil off the surface of the water.

In a third embodiment, a device for extracting oil from food waste is provided. The device comprises an elongated cylinder disposed in a reservoir and being at least partially immersed under water, the cylinder comprising a proximate end and a distal end, the food waste being introduced to the proximate end; at least one pore at the distal end, the at least one pore being on a sidewall; a steam inlet at the distal end that introduces steam into the cylinder; a drive shaft that extends the length of the cylinder; and a motor for actuating the drive shaft while the water is at a temperature of at least 80° C. and steam is being supplied to the cylinder through the steam inlet, the drive shaft simultaneously pressing the food waste against an interior wall of the cylinder and moving the food waste from the proximate end to the distal end, thereby extracting an oil from the food waste.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses otherequally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic cross section of one device for extracting oil from food waste;

FIG. 2A is a schematic cross section of another device for extracting oil from food waste;

FIG. 2B is an end view of a cylinder of a device for extracting oil from food waste wherein a hatch is illustrated;

FIG. 2C is a schematic cross section of yet another device for extracting oil from food waste;

FIG. 3 is a schematic cross section of yet another device for extracting oil from food waste;

FIG. 4 is a graph depicting glucose concentration in various food waste samples as a function of time;

FIG. 5 is a graph depicting ethanol concentration in various food waste samples as a function of time; and

FIG. 6 is a graph depicting ethanol concentration for different food waste samples with and without treatment with α-amylase.

### DETAILED DESCRIPTION OF THE INVENTION

The disclosed method uses municipal garbage as a plentiful, renewable, and carbon-rich feedstock. The disclosed device and process addresses the dual problems of renewable energy and disposal of the organic component of municipal solid waste (MSW). Specifically, the method extracts oil from food waste and breaks down the non-oily material to a slurry or aqueous suspension, leaving behind material such as bone, cellulose, and lignin. The oil is used to make biodiesel or other biofuels, and the slurry is suitable for fermentation to ethanol or anaerobic digestion to biogas. Alternatively, the non-oily material, including any bone, cellulose, or lignin is suitable for composting, as most of the fats, oils, and greases (FOG) which attract animal vectors have been removed in the process.

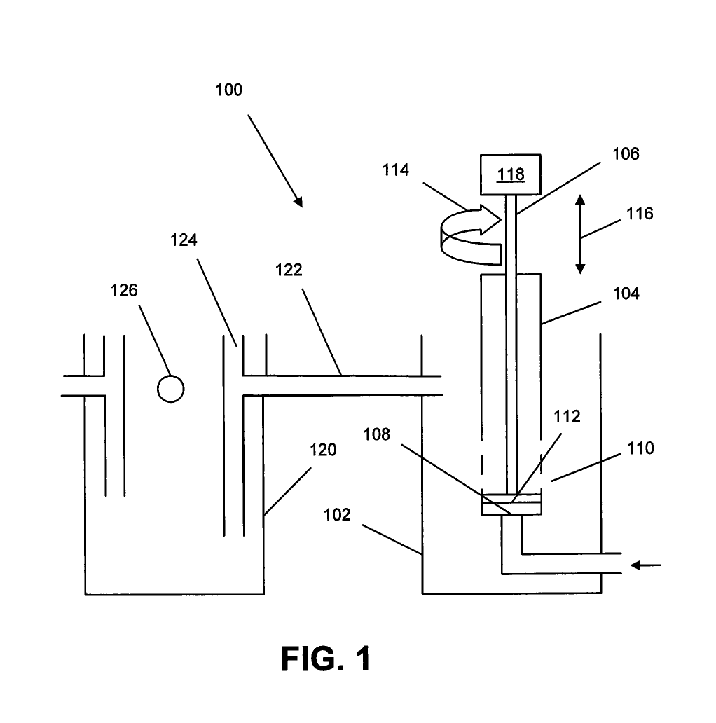

An exemplary device 100 is schematically illustrated in FIG. 1. The device 100 comprises a reservoir 102, a porous cylinder 104 with a rotating and reciprocating drive shaft 106, and a steam inlet 108 at the bottom of the cylinder. The porous cylinder 104 has pores 110. The drive shaft moves a disc 112 in a rotary direction 114 and in a reciprocating direction 116. The piston shaft is connected to a variable speed drill motor, operating between 0 and 100 rpm. The piston uses between 2 and 5 seconds to be lowered and raised while rotating, which presses the waste slurry through the holes in the cylinder. Reloading the cylinder takes about 10 seconds. The food waste is placed in the porous cylinder 104 and the drive shaft 106 is inserted. In another embodiment, the drive shaft 106 is equipped with an inlet that provides a continuous on-demand feed of food waste. If the food waste contains large objects such as cans, bottles, metal objects, large bones, etc., the food waste may be screened to remove those objects. A steam hose is fitted to the steam inlet 108 at the bottom of the porous cylinder and the assembly is immersed in the reservoir 102 which is filled with hot water. The water may be heated to a temperature of between 80° C. and 100° C. with an external heating element or by the steam, or a combination of both. A motor 118 is connected to the drive shaft 106 and the rotating disc 112 is moved up and down in the porous cylinder 104. The reciprocating motion of the disc 112 squeezes the food waste and releases the oil, which floats to the top of the water reservoir 102. The oil collects in grease trap 120 by way of port 122 where it is collected for conversion to biodiesel or other uses. A vertical path 124 is used to trap oils in the grease trap 120 and prevent backflow of the oils. The rotary motion of the disc 112 helps to mechanically break down the food waste. The soluble or suspended components exit the cylinder 104 through the pores 110, while most of the bone, cellulose, and lignin components remain in the cylinder 104 for later removal and disposal. An oil exit port 126 is provided proximate the top of the grease trap 120 for collecting oil that is floating on the surface of the water. An important advantage of this process is that the suspended food particles either remain suspended or settle to the bottom of the container, where they can be collected for fermentation or anaerobic digestion. If small food particles coated with oil float to the top, they can be screened to prevent movement into the grease trap. Without this treatment much of the food waste floats to the top of the vessel and interferes with oil collection.

The disclosed method exploits an unexpected benefit provided by the combination of hot water treatment, steam treatment and pressing. The comparative examples provided elsewhere in this specification show that treatment with hot water, steam or pressing alone removed only a small portion of the oil. The combination of hot water treatment, steam treatment and pressing also breaks down the food waste so the resulting slurry is better suited for fermentation or anaerobic digestion.

The process meets the need of extracting usable fuel precursors from waste material that would otherwise be incinerated or disposed in a land fill. The oil can be processed to biodiesel, and the non-oily, starchy material can be fermented to ethanol. Alternatively, it can be anaerobically digested to biogas.

The device allows the process to be accomplished more rapidly, compared to the soaking and/or steaming and manual pressing. The device also provides better odor control compared to the manual steaming and pressing process. Rapid processing of municipal solid waste is necessary due to the large volume of material produced, and potential problems with odor control. The device was designed to be scalable, so that a small version can be built for small waste producers, and larger versions built using the same principles for large operators such as departments of sanitation, land fill operators, recycling facilities, etc. Commercially, the use of organic solvents to extract oil is undesirable due to volatile organic compounds (VOC) emissions and a desire to replace petroleum as a source of fuel and chemicals.

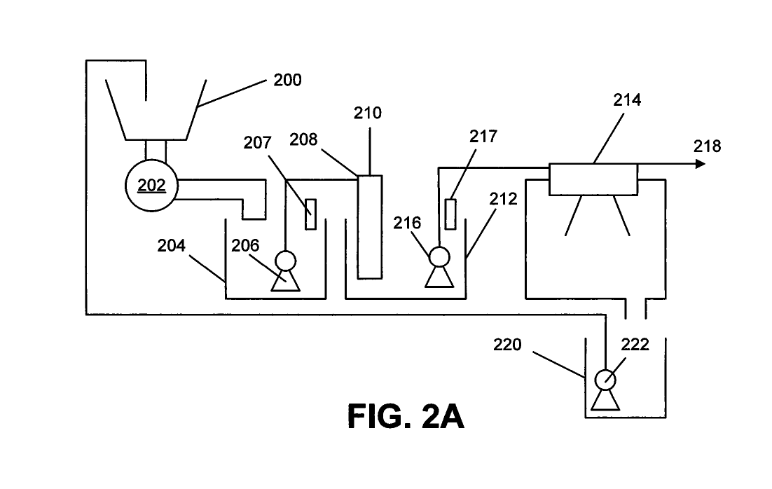

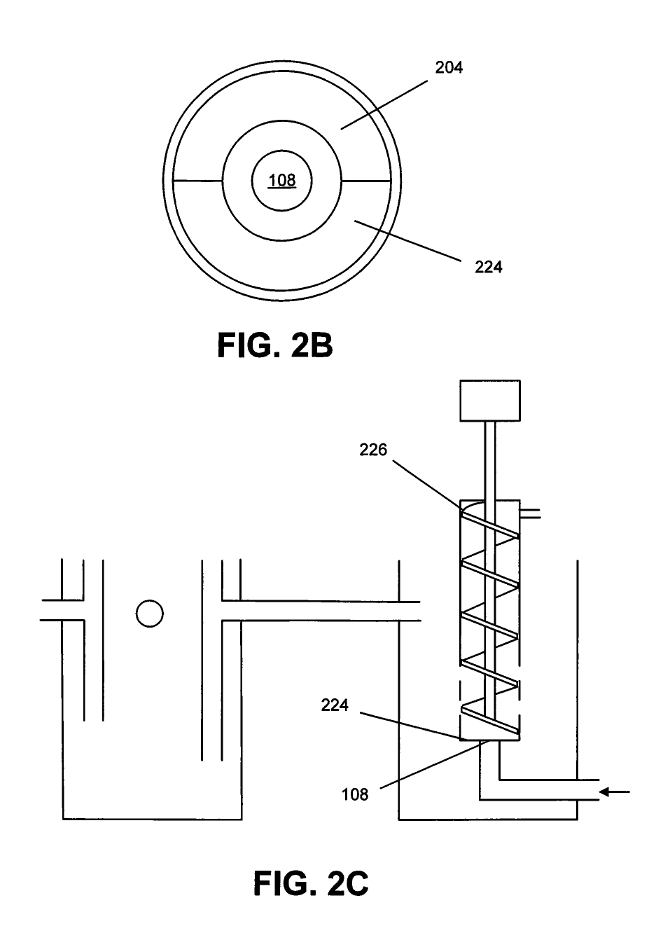

In other embodiments, a garbage grinder adds grinding of the waste to the process. The waste-water slurry enters the device via an intake port 200 (see FIG. 2A) below the top of the disc travel. In the embodiment of FIG. 2A a first sink 200 introduces food waste into a garbage grinder 202 which empties into a second sink 204. An oil skimmer 207 removes oil from the second sink 204 and, in one embodiment, supplies the oil to centrifuge 214. A pump 206 coveys the food waste into an intake port 208 in the cylinder 210. Extracted oils accumulate in the reservoir 212. An oil skimmer 217 removes oil from the reservoir 212 similar to the oil skimmer 207. The oil/water mixture is then transferred to centrifuge 214 by pump 216. Oils are collected by exit port 218 while water from the centrifuge 214 are collected in third sink 220 and subsequently transferred back to the first sink 200 with a pump 222.

In still other embodiments, a means for removing remaining waste from the cylinder without having to disassemble the apparatus is provided. For example, a hatch 224 (see FIG. 2B) may be provided in the bottom of the cylinder or in a sidewall of the cylinder proximate the bottom. The hatch may be opened, either automatically at a predetermined schedule, or manually by a user, to remove remaining waste from the cylinder. An exit port with a valve and suction is also contemplated. In certain applications, small starch particles at the bottom of the first bucket are easily agitated and float to the top. A submersible pump (e.g. pump 206) that can handle solids, or a wet-dry vacuum, may be used to remove those particles and remove the particles using a filter 204 for eventual fermentation. The filtered water may be returned the device. Referring to FIG. 2C, in one embodiment, a rotating auger 226 replaces the rotating disc 112.

A first-generation device was prepared from a grease trap equipped with an immersion heater and a cloth bag to hold the food waste. The initial version of the device utilized manual pressing to remove oil. The bag was immersed in the water at about 80° C., and pressed with a flat piece of metal until no more oil was observed rising to the top of the grease trap. This process took approximately one hour. No steam was used at this point, and the oil yields are less consistent and generally lower, compared to the steam apparatus described elsewhere in this specification. Several types of food waste were soaked and pressed in this manner, as shown in Table 1.

TABLE 1
Food waste oil extraction by manual pressing in 80° C. water.

| | | | | | | % total |
|---|---|---|---|---|---|---|
| | | | Oil | | | oil in |
| | | Oil water | solvent | Total | % oil | sample |
| Mass | | extraction | extraction | oil | removed | (wet |
| (kg) | Description | (g) | (g) | (g) | by water | mass) |
| 1.0 | Chicken | 26.2 | 33.7 | 59.9 | 43.7 | 6.0 |
| | meat | | | | | |
| | and bones | | | | | |
| 1.0 | Chicken | 16.3 | 21.4 | 37.7 | 43.2 | 3.8 |
| | meat | | | | | |
| | and bones | | | | | |
| 1.0 | Chicken | 49.7 | 28.8 | 78.5 | 63.3 | 7.8 |
| | meat | | | | | |
| | and bones | | | | | |
| 1.0 | Spent coffee | 0 | 34.6 | 34.6 | 0 | 3.4 |
| | grounds | | | | | |
| 1.0 | Spent coffee | 0 | 10.5 | 10.5 | 0 | 1.0 |
| | grounds | | | | | |
| 1.0 | Spent coffee | 0 | 11.1 | 11.1 | 0 | 1.1 |
| | grounds | | | | | |
| 1.0 | Rice and | 6.6 | 14.3 | 20.9 | 31.7 | 2.1 |
| | vegetables | | | | | |
| 1.0 | Vegetables | 0 | 0 | 0 | N/A | 0 |
| 1.0 | Vegetables | 0 | 0 | 0 | N/A | 0 |
| 3.0 | Meat, | 50.3 | 10.7 | 61.0 | 82.4 | 2.0 |
| | vegetables, | | | | | |
| | bones | | | | | |
| 2.0 | Meat, | 130.3 | 15.3 | 145.6 | 89.5 | 7.3 |
| | vegetables, | | | | | |
| | bones | | | | | |
| 1.5 | Meat, | 62.5 | 10.8 | 73.3 | 85.3 | 4.9 |
| | vegetables, | | | | | |
| | bones | | | | | |

The manual soak and squeeze sequence proved that these steps can remove a significant portion of the oil, but it is much too labor intensive to be practical on a large scale. An improved method was therefore necessary. Steam was used in other experiments that used a crude apparatus that was very labor intensive, described elsewhere in this specification. Thus, experiments have been done with and without steam, and steam was more efficient.

It was soon realized that pressing or squeezing the waste was necessary to remove the oil-water emulsion, and that steam removed the oil more efficiently than hot water alone. An apparatus was built consisting of a grease trap, steam generator, water heater, and a nylon stocking to hold the waste, which was suspended from a ring stand and clamp. The apparatus is shown in FIG. 1. The stocking was filled with food waste, immersed in the hot water at about 80° C., removed from the water, and blasted with steam before manually squeezing out the oil-water emulsion into the grease trap. The process was repeated until the liquid no longer appeared oily. The solid was then removed from the stocking, dried, and the remaining oil extracted with hexane. Approximately 85% of the oil was removed by the hot water, steam, and squeezing sequence.

To address the scalability problem, a second-generation design was chosen in which ground food waste is fed into a 7.6 cm diameter metal cylinder with parallel rows of holes (approximately 3.2 mm in diameter) drilled in the bottom, through which the ground waste can be extruded. In one embodiment, the pores are at least 3 mm in diameter and less than 50 mm in diameter. In another embodiment, the pores at least 3 m in diameter and less than 10 mm in diameter. A steam inlet was fitted to a plug at one end of the cylinder. A rotating disc was attached to a threaded steel rod, and a drill was used to rotate the drive shaft while manually moving it up and down in the cylinder until the waste was extruded through the holes, at which point the cylinder was reloaded. The rotation of the disc helped to loosen the oil from the waste particles, and upon extrusion, the oil floated to the top of the water container in which the device was immersed. The oil was manually skimmed from the water in which the cylinder assembly was immersed, and the solids settled to the bottom. The solids were subsequently pumped to a continuous centrifuge fitted with a steam inlet to maximize the oil removal. The separated liquid was collected in a holding tank, and any remaining oil was manually skimmed from the top. The results are shown in Table 2. This manual skimming was labor intensive, and there was some oil lost in the process.

TABLE 2
Food waste oil extraction using manual oil skimming.

| | Oil | Oil | Total | | % total |
|---|---|---|---|---|---|
| | extracted | extracted | oil in | % oil | oil in |
| Mass | with steam | with solvent | sample | removed | sample (wet |
| (kg) | (g) | (g) | (g) | by steam | mass) |
| 2.564 | 21.986 | 4.915 | 26.901 | 81.7 | 1.05 |
| 2.056 | 54.661 | 26.644 | 81.305 | 67.2 | 3.95 |
| 2.450 | 20.616 | 3.897 | 24.513 | 84.1 | 1.00 |
| 2.400 | 34.070 | 9.505 | 43.575 | 78.2 | 1.82 |
| 3.072 | 33.933 | 4.082 | 38.015 | 89.3 | 1.24 |
| 2.888 | 76.779 | 1.269 | 78.048 | 98.4 | 2.70 |
| 3.324 | 22.102 | 1.552 | 23.654 | 93.4 | 0.712 |
| 3.306 | 19.320 | 3.252 | 22.572 | 85.6 | 0.683 |
| 3.072 | 16.038 | 2.062 | 18.100 | 88.6 | 0.589 |
| 3.636 | 16.857 | 11.319 | 28.176 | 59.8 | 0.775 |
| Average | | | | 82.6 | 1.45 |
| Std. Dev. | | | | 11.7 | 1.09 |

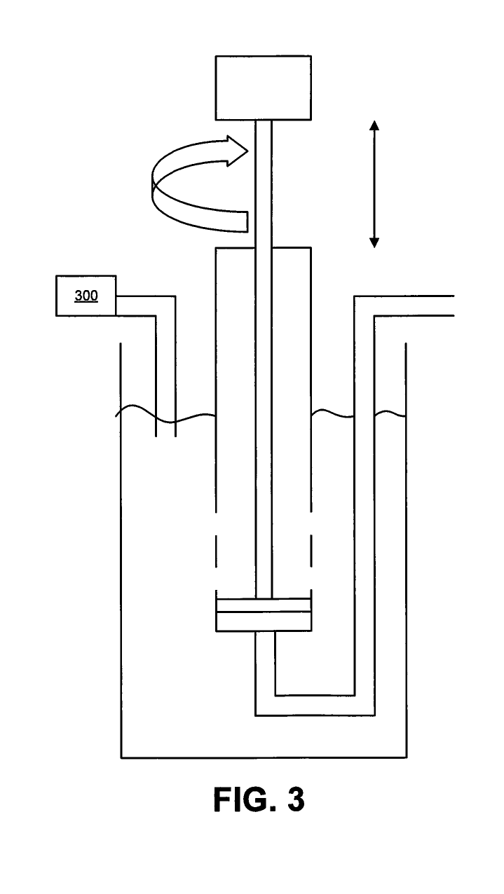

This process was enhanced in a third-generation device by adding a pair of rotating disk oil skimmers 300, and by pumping the water from the centrifuge outlet back into the garbage inlet sink, thus closing the loop and recycling all of the waste water. The centrifuge is one designed for cleaning biodiesel. It is continuous in operation, but the bowl should be cleaned periodically as it fills up with solids. The oil skimmers were placed in the waste slurry collection tank after the garbage grinder, and in the tank in which the extrusion cylinder was immersed. The entire apparatus is shown schematically in FIG. 3.

The enhanced process resulted in improved oil extraction, as shown by the data in Table 3. In most cases the oil removed by steam was greater than 95% of the total oil recovered (i.e. only an additional 5% oil could be removed by sequent extraction of the dried solids with hexane). The exceptions occurred in samples with very low oil content. The overall average oil recovery with the steam, extrusion, and skimming process was 72.2%, but that was increased to 96.2% in samples with at least 3.0% oil by wet mass. On average, the oil yield was 29.2 g oil/kg garbage based on the wet mass. This is about one half of the hydrocarbon generation potential of Northeast China oil shales of 59-88.2 g hydrocarbons/kg rock. However, municipal garbage is much easier to obtain than oil shale, and the solid byproduct is also a potential fuel precursor, as described below. The treatment time varied, but the total treatment time averaged about 2-3 hours to process one batch. That included setup, break down, cleanup, and cleaning of spills.

TABLE 3
Food waste oil extraction using rotating disk oil skimmer.

| | Oil | Oil | Total | | % total |
|---|---|---|---|---|---|
| | extracted | extracted | oil in | % oil | oil in |
| Mass | with steam | with solvent | sample | removed | sample (wet |
| (kg) | (g) | (g) | (g) | by steam | mass) |
| 3.246 | 101.410 | 5.091 | 106.501 | 95.2 | 3.28 |
| 3.365 | 151.384 | 2.406 | 153.790 | 98.4 | 4.57 |
| 3.186 | 52.514 | 3.156 | 55.670 | 94.3 | 1.74 |
| 1.920 | 3.427 | 7.145 | 10.572 | 32.4 | 0.551 |
| 2.280 | 153.020 | 5.255 | 158.275 | 96.7 | 6.94 |
| 2.466 | 79.208 | 1.677 | 80.885 | 97.9 | 3.28 |
| 2.516 | 24.358 | 17.625 | 41.983 | 58.0 | 1.67 |
| 3.046 | 1.781 | 2.512 | 4.293 | 41.5 | 0.141 |
| 2.788 | 56.136 | 10.272 | 66.408 | 84.5 | 2.38 |
| 3.040 | 131.346 | 10.191 | 141.537 | 92.8 | 4.66 |
| Overall | | | | 79.2 | 2.92 |
| Average | | | | | |
| Overall | | | | 25.3 | 2.07 |
| Std. Dev. | | | | | |
| Average | | | | 96.2 | 4.54 |
| >3.0% oil | | | | | |
| Std. Dev. | | | | 2.3 | 1.50 |
| >3.0% oil | | | | | |
| Average | | | | 62.2 | 1.20 |
| <3.0% oil | | | | | |
| Std. Dev. | | | | 26.8 | 0.923 |
| <3.0% oil | | | | | |

The third-generation device was further refined, resulting in the device illustrated in FIG. 1.

After oil extraction, the solids may be fermented. An exemplary hydrolysis process was done in a 500 m1 conical flask; 100 g of food waste was added with 100 g of distilled water to make a solid liquid ratio of 1:1. During liquefaction, 200 μl of alpha-amylase (Type XII-A, bacterial, from bacillus licheniformis, from Sigma Aldrich to provide 3225.2 units of alpha-amylase to the food waste during liquefaction) was added to the food waste sample and kept at 65° C. for 1 hour stirring at 150 rpm. The initial pH of the sample was adjusted to 5.5 due to the optimum pH value for alpha amylase activity is between 5.0 and 6.0 with sulfuric acid. Every 24 hours during the fermentation process, 10 m1 of the sample was collected from the aqueous phase for glucose and ethanol concentration analyses. The sample was centrifuged at 7800 rpm for 15 minutes and the supernatant was filtered through a syringe filter with pore size of 0.45 μm. Both glucose and ethanol concentrations were analyzed by using high performance liquid chromatography equipped with refractive index detector. A BIO-RAD HPX-87H column was used at 65° C. with 0.0005 M sulfuric acid solution as mobile phase. The flow rate of the system was set to be 0.6 mL per min. Glucose and ethanol standard curves were prepared following a published procedure (Shimadzu, Using the Shimadzu HPLC System in the Fuel-Grade Ethanol Production Laboratory, Oct. 20, 2007). In order to ease the enzymatic process, the sample was fermented without excess heating or receiving any additional pre-treatment. 10% v/v of the yeast S. cerevisiae was added to the hydrolyzed sample. Sulfuric acid (0.1N) was added to the sample to adjust the pH to the optimum value of 4.5, and the flask was placed in a shaking incubator for 96 hours. The temperature and agitation speed for the whole fermentation process was set to be consistent at 30° C. and 150rpm. Another set of food waste fermentation was also carried out without treatment with enzymes prior to addition of the yeast S. cerevisiae which served as the control experiment.

Generally, these food wastes collected included meat, pasta, rice, potato, bread and some peelings and parts of fruits and vegetables. Samples I and II were mixtures of rice, meat, fish, and vegetables. Food waste III contained mainly rice and other food waste which were rich in starch. For food waste IV, it contained approximately 80% of vegetable and fruit peels.

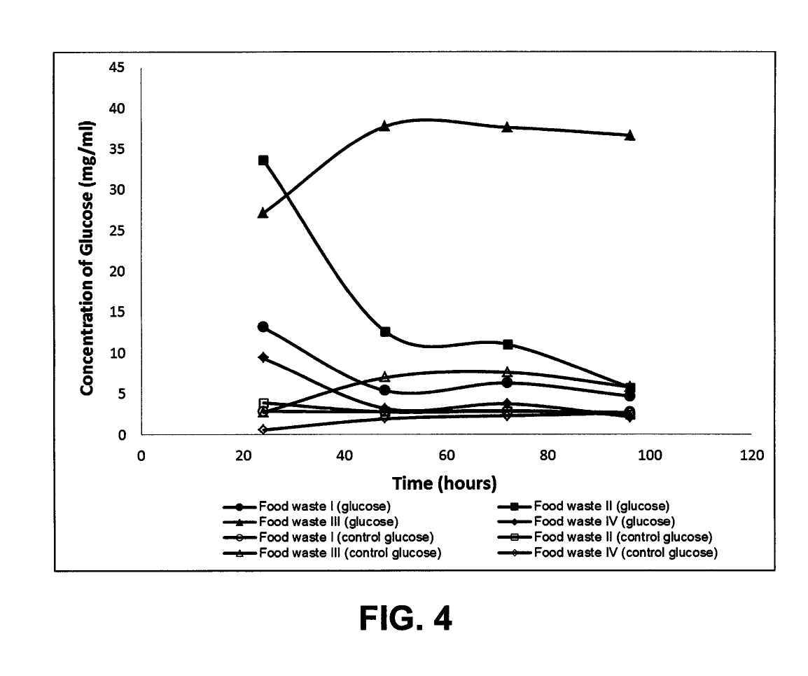

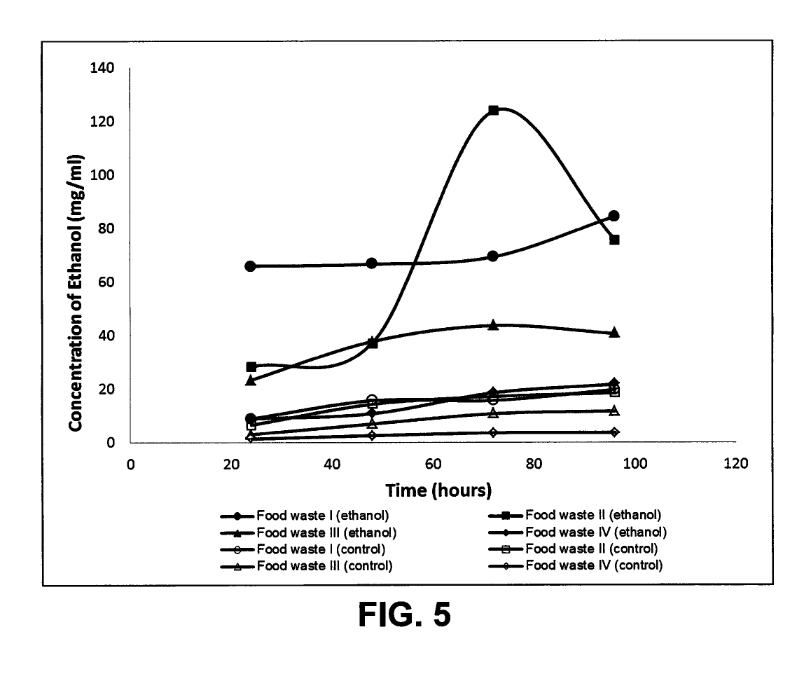

The glucose concentrations as a function of time for four treated and four control samples are shown in FIG. 4, and the corresponding ethanol concentrations are shown in FIG. 5. FIG. 4 depicts the concentration of glucose produced after saccharification by using α-amylase for four different food wastes against time. Readings are taken from average of duplicate analyses of food waste. FIG. 5 depicts the concentration of ethanol produced after saccharification and fermentation by using aα-amylase and S. cerevisiae yeast for four different food wastes against time. Readings are taken from average of duplicate analyses of food waste.

FIG. 4 and FIG. 5 summarize the results of glucose and ethanol concentrations from saccarification and fermentation of four different food wastes. On average, the glucose production by α-amylase during saccarification is considered to be completed prior to fermentation. This can be seen from the decreasing trend of glucose concentration with time in FIG. 4, in which the glucose has slowly been converted to ethanol in the fermentation process with increasing reaction time. However, the highest glucose concentration attained for the experiment is only around 37 mg per ml . . .

In contrast to the glucose production, ethanol concentration is showing an increasing trend as the time increases from FIG. 5. Since food waste samples I and II are different mixtures of food waste, the composition of carbohydrate to protein and other food class in the waste sample might be different. Hence the ethanol concentration generated is shown to give nearly 50% different throughout the experimental timeframe. This can also be proven from the glucose recovery, in which the glucose content in food waste I is lower than that of food waste II. A significant drop of ethanol production after 72 hours was observed for food waste II. This is an unusual observation than the normal trend because the concentration of ethanol should increase but not decrease.

Food waste III contained mainly rice and other food waste which were rich in starch. By applying the food waste III with α-amylase in which the starch conversion to glucose was expected to be high, the results turned out to be the opposite. Glucose concentration analysed only recorded to be around 36 mg/ml, leading to low concentration of ethanol (41 mg/ml) at 96 hours. For food waste IV, although it contained approximately 80% of vegetable and fruit peels, α-amylase was still noticed to be able to hydrolyse the remaining 20% of non-fibre waste that might contain starchy waste such as potatoes. Therefore, there is a little amount of ethanol produced up to 22 mg/ml in 96 hours.

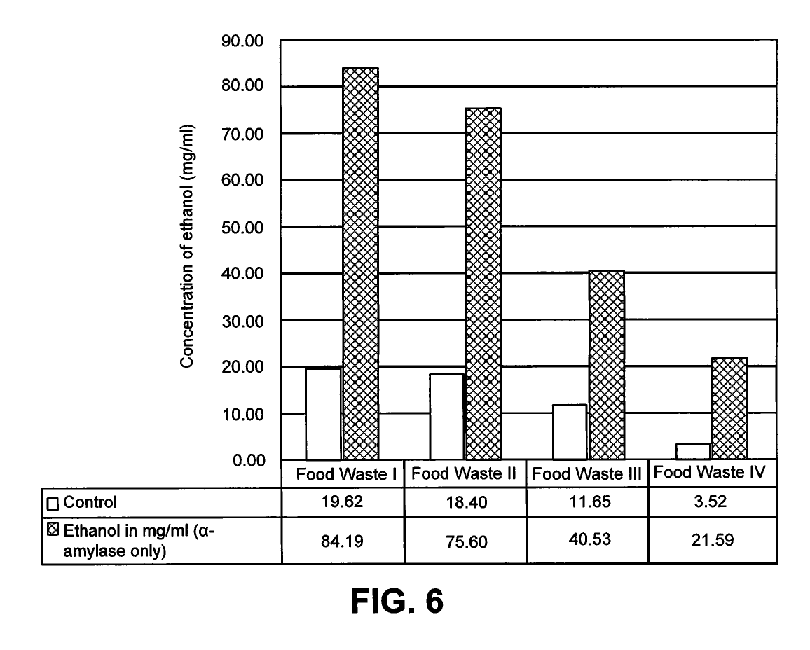

A comparison of the overall ethanol yield after 96 hours is shown in FIG. 6 for all food waste types. When comparing with all the control samples, there is a significant increase in glucose recovery and ethanol production with the addition of α-amylase enzyme during hydrolysis of food wastes prior to fermentation. When comparing with control samples, the average concentration of ethanol produced by using α-amylase after 96 hours for food waste I increased from 19.62 mg/ml to an average of 84.19 mg/ml. Besides than food waste I, the other three types of wastes also show the similar trend. For food waste II, the ethanol production increased from 18.40 mg/ml to 75.60 mg/ml when α-amylase was added in. For food waste III, the ethanol production does not increased much when comparing to food wastes I and II. It only increased four times of the ethanol produced in the control sample when α-amylase was added in. Finally for food waste IV, the ethanol produced without any enzymatic hydrolysis is very low. When α-amylase was added to the samples, the ethanol production increased from 3.52 mg/ml to 21.59 mg/ml.

The oil extracted from food waste may be referred to as “orange grease” because it frequently has an orange color. Orange grease can be converted to biodiesel by a previously known acid catalyzed process. See Pratt et al. Journal of Science & Technology Development (Vietnam) 2012, 15, 47-56 “Food Waste as a Source of Biofuels Processes” has also been developed to convert brown grease to a hydrocarbon kerosene-like fuel. See Pratt et al. NEWEA Journal 2014, 48(2), 44-53, “Beneficial Use of Brown Grease—A Green Source of Petroleum Derived Hydrocarbons”. To determine whether that procedure is also applicable to orange grease, three pyrolysis experiments were performed in parallel. Samples of brown grease (approximately 40 g), orange grease, and a 50:50 mixture of brown greases were pyrolyzed. One set of three trials was performed without an added catalyst, and a second set of 3 trials was performed with 100 mg Fe₂(SO₄)₃, according to our previously published procedure. The results are shown in Table 4. The yields of distillate, water, and solid residue are reported as a percentage of the original sample mass. The distillate consists essentially of alkane and alkene hydrocarbons (the desired products). Water is a waste byproduct. The solids consist essentially of char, which may be a waste product, but it can potentially be gasified and converted to useful products. Although the distillate yields appear to be higher without the catalyst, they were found to contain unreacted free fatty acids (FFA), which is undesirable in fuel.

TABLE 4
Pyrolysis results of orange, brown,
and orange-brown blended greases.

| | | % yield | % yield | % yield | |
|---|---|---|---|---|---|
| Set | Grease | distillate | water | solids | Catalyst |
| 1 | Orange | 63.0 | 4.94 | 10.2 | Fe₂(SO₄)₃ |
| 1 | Blend | 65.6 | 4.72 | 9.25 | Fe₂(SO₄)₃ |
| 1 | Brown | 63.5 | 4.75 | 11.7 | Fe₂(SO₄)₃ |
| 2 | Orange | 65.1 | 5.12 | 9.80 | Fe₂(SO₄)₃ |
| 2 | Blend | 61.2 | 4.12 | 10.8 | Fe₂(SO₄)₃ |
| 2 | Brown | 65.9 | 6.24 | 10.4 | Fe₂(SO₄)₃ |
| 3 | Orange | 69.8 | 4.55 | 8.90 | Fe₂(SO₄)₃ |
| 3 | Blend | 68.1 | 4.58 | 7.90 | Fe₂(SO₄)₃ |
| 3 | Brown | 65.8 | 4.66 | 11.4 | Fe₂(SO₄)₃ |
| 4 | Orange | 73.6 | 2.52 | 10.2 | none |
| 4 | Blend | 76.5 | 2.45 | 4.99 | none |
| 4 | Brown | 68.4 | 4.73 | 23.7 | none |
| 5 | Orange | 70.4 | 3.40 | 6.85 | none |
| 5 | Blend | 67.6 | 1.90 | 10.1 | none |
| 5 | Brown | 69.2 | 4.27 | 9.61 | none |
| 6 | Orange | 73.2 | 0.966 | 6.40 | none |
| 6 | Blend | 75.8 | 1.32 | 3.90 | none |
| 6 | Brown | 78.4 | 2.85 | 7.87 | none |

In summary, the ethanol yield increases upon the addition of α-amylase into the system. Mixture of food waste I and food waste II showed a higher ethanol production because in food waste, rice is not the only starchy materials that are able to be hydrolyzed into glucose; potatoes, corn and bread can also be hydrolyzed into simpler monomeric sugar. In contrast, food wastes that contained mostly vegetable (food waste IV) was unable to produce high amount of ethanol because cellulose present in the plant cell does not contain much starch. After all, it still can be used as a waste source to produce ethanol, which is a value-added commodity.

Experimental

As previously discussed, the disclosed method exploits an unexpected benefit provided by the combination of hot water treatment, steam treatment and pressing. The comparative examples provided below show that treatment with hot water, steam or pressing alone removed only a small portion of the oil.

Preparation of the food wastes: Collected food wastes were separated into different containers. Meat bones, plastics and glass pieces were removed from the waste sample. The remaining food waste samples were ground into small pieces suitable for saccharification process. This was ground in a blender. The pieces were generally less than 5 mm. The separated waste samples were stored in a refrigerator at 4° C. Generally, the food waste collected included meat, pasta, rice, potato, bread and peelings and parts of fruits and vegetables.

### EXAMPLE 1

### Extraction with Boiling Water—One Hour.

654 grams of food waste (not ground) was placed in a cotton bag. The cotton bag was immersed in a one liter beaker of boiling water for one hour. After one hour, the bag was removed and allowed to drain into the beaker, but the bag was not squeezed or pressed to remove additional oil. The contents of the beaker were cooled and transferred to a separatory funnel to collect the extracted oil. After draining the waste was dried and extracted with hexane to remove the remaining oil. Oil extracted with hot water: 0.166 g; Oil extracted with hexane: 2.420 g (6.42% recovery). Conclusion: Hot water alone is not efficient in removing oil from food waste.

### EXAMPLE 2

### Extraction with Boiling Water—Ten Hours.

Food waste (not ground) was placed in a nylon stocking and stocking placed in 85° C. water for 10 hours. The process of Example 1 was repeated. 59% of the total oil was recovered. Prolonged treatment increased oil recovery but overall efficiency was still low.

### EXAMPLE 3

### Extraction with Boiling Water after Grinding—One Hour.

Example 1 was repeated, but with the food waste being subjected to grinding in blender prior to placing in stocking. 30% of the oil was recovered. Grinding food waste resulted in only a modest increase in oil recovery with overall poor efficiency.

### EXAMPLE 4

### Extraction with Boiling Water after Grinding—One Hour.

Example 1 was repeated with ground food waste, but soaking was combined with steaming from steam generator and manually squeezing the stocking to remove the oil-water mixture. The steam was intermittently (approximately 1-2 minutes steam per 5 minutes soaking in hot water) passed through the water during the extraction. 85% of the oil was recovered by this method.

### EXAMPLE 5

### Extraction of Food Waste Oil with Steam.

102 grams of food waste was placed on a screen above a 400 mL beaker of boiling water for one hour. The beaker contents, containing water and oil from the food on the screen was cooled and transferred to a separatory funnel to collect the oil. The food waste was then dried and extracted with hexane to remove the remaining oil. Oil extracted with steam: Trace (less than 100 mg); Oil extracted with hexane: 3.055 g; Conclusion: Steam treatment alone does not remove a significant percentage of the oil.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for extracting oil from food waste, the method comprising steps of:
introducing food waste into an elongated cylinder that has a length, the elongated cylinder disposed in a reservoir and being at least partially immersed under water, the cylinder comprising:
a proximate end and a distal end, the food waste being introduced to the proximate end;
at least one pore at the distal end, the at least one pore being on a sidewall;
a steam inlet at the distal end that introduces steam into the cylinder;
a drive shaft that extends the length of the cylinder;
actuating the drive shaft with a motor while the water is at a temperature of at least 80° C. and steam is being supplied to the cylinder through the steam inlet, the drive shaft simultaneously pressing the food waste against an interior wall of the cylinder and moving the food waste from the proximate end to the distal end, thereby extracting an oil from the food waste;
extruding the oil through the at least one pore, the oil thereafter floating to a surface of the water;
skimming the oil off the surface of the water.

2. The method as recited in claim 1, wherein the drive shaft has a disc at a terminal end, the drive shaft being configured to simultaneously rotate the disc and cause the disc to reciprocate over a portion of the length of the cylinder.

3. The method as recited in claim 1, wherein the cylinder further comprises a hatch at the distal end of the cylinder, the method further comprising a step of opening the hatch and removing food waste.

4. The method as recited in claim 3, wherein the method is a batch method and the method is stopped and hatch is opened after less than two hours and food waste is removed from the cylinder.

5. The method as recited in claim 1, wherein at least 80% of total oil is recovered after one hour of treatment by the method as determined by extracting residual food waste with hexane, when the food waste is at least 3.0% oil by wet mass.

6. The method as recited in claim 1, wherein at least 90% of total oil is recovered after one hour of treatment by the method as determined by extracting residual food waste with hexane, when the food waste is at least 3.0% oil by wet mass.

7. The method as recited in claim 1, wherein the at least one pore has a diameter of at least 3 mm.

8. The method as recited in claim 1, wherein the at least one pore has a diameter of at least 3 mm and less than 50 mm.

9. The method as recited in claim 1, wherein the at least one pore has a diameter of at least 3 mm and less than 10 mm.

10. A method for extracting oil from food waste, the method comprising steps of:
introducing food waste into an elongated cylinder that has a length, the elongated cylinder disposed in a reservoir and being at least partially immersed under water, the cylinder comprising:
a proximate end and a distal end, the food waste being introduced to the proximate end;
at least one pore at the distal end, the at least one pore being on a sidewall;
a steam inlet at the distal end that introduces steam into the cylinder;
a drive shaft that extends the length of the cylinder;
actuating the drive shaft with a motor while the water is at a temperature of at least 80° C. and steam is being supplied to the cylinder through the steam inlet, the drive shaft simultaneously pressing the food waste against an interior wall of the cylinder and moving the food waste from the proximate end to the distal end, thereby extracting an oil from the food waste, wherein the drive shaft has an auger, the drive shaft being configured to rotate the auger and move the food waste from the proximate end to the distal end
extruding the oil through the at least one pore, the oil thereafter floating to a surface of the water;
skimming the oil off the surface of the water.

11. The method as recited in claim 10, the cylinder further comprising an intake port at the proximate end for receiving the food waste.

12. The method as recited in claim 11, wherein the method is a continuous method.

13. The method as recited in claim 10, wherein the at least one pore has a diameter of at least 3 mm and less than 50 mm.

14. The method as recited in claim 10, wherein at least 80% of total oil is recovered after one hour of treatment by the method as determined by extracting residual food waste with hexane, when the food waste is at least 3.0% oil by wet mass.

15. A device for extracting oil from food waste, the device comprising:
an elongated cylinder disposed in a reservoir and being at least partially immersed under water, the cylinder comprising:
a proximate end and a distal end, the food waste being introduced to the proximate end;
at least one pore at the distal end, the at least one pore being on a sidewall;
a steam inlet at the distal end that introduces steam into the cylinder;
a drive shaft that extends the length of the cylinder;
a motor for actuating the drive shaft while the water is at a temperature of at least 80° C. and steam is being supplied to the cylinder through the steam inlet, the drive shaft simultaneously pressing the food waste against an interior wall of the cylinder and moving the food waste from the proximate end to the distal end, thereby extracting an oil from the food waste.

16. The device as recited in claim 15, wherein the drive shaft has a disc, the drive shaft being configured to simultaneously rotate the disc and cause the disc to reciprocate over a portion of the length of the cylinder.

17. The device as recited in claim 15, wherein the drive shaft has an auger, the drive shaft being configured to rotate the auger and move the food waste from the proximate end to the distal end.
